(19) United States
(12) Patent Application Publication
(10) Pub. No.: US 2013/0143857 A1
(43) Pub. Date: Jun. 6, 2013

(54) PHARMACEUTICAL COMPOSITIONS OF CEFDITOREN PIVOXIL

(71) Applicants: Ashish Guha, Pune (IN); Bharat Metkar, Pune (IN); Makrand Avachat, Pune (IN)

(72) Inventors: Ashish Guha, Pune (IN); Bharat Metkar, Pune (IN); Makrand Avachat, Pune (IN)

(73) Assignee: LUPIN LIMITED, Mumbai, State of Maharashtra (IN)

(21) Appl. No.: 13/810,919

(22) PCT Filed: Jul. 14, 2011
(86) PCT No.: PCT/IB11/01635

§ 371 Date: Jan. 18, 2013

(30) Foreign Application Priority Data
Jul. 23, 2010 (IN) 807/KOL/2010

(51) Int. Cl. A61K 31/546; A61K 31/545

(52) U.S. Cl. CPC A61K 31/546
USPC 514/206

(57) ABSTRACT

The present invention relates to a pharmaceutical composition comprising Cefditoren pivoxil, water soluble high molecular weight substance and one or more pharmaceutically acceptable excipient wherein weight ratio of high molecular weight substance to Cefditoren pivoxil is greater than 1:4 and a process for preparation thereof.

### FIELD OF THE INVENTION

The present invention relates to pharmaceutical compositions of Cefditoren pivoxil and a process for preparation thereof.

### BACKGROUND OF THE INVENTION

Cefditoren is a cepham compound: 7-[2-methoxyimino-2-(2-aminothiazol-4yl)acetamido]-3-[2-(4-methylthiazol-5-yl)vinyl]-3-cephem-4-carboxylicacid (synisomer, cis-isomer). Cefditoren synthesis is disclosed in U.S. Pat. Nos. 4,839,350 and 4,918,068

It is generally known that Cefditoren, when used in an oral preparation, has a very broad antimicrobial spectrum while enjoying low toxicity and is very useful for the therapy and prophylaxis of diseases induced by gram-positive and gram-negative bacteria as disclosed in Japanese Patent Publication No. 64503/1991, U.S. Pat. No. 4,839,350 and European Patent No. 175610.

Cefditoren pivoxil is a novel prodrug in which a pivaloyloxymethyl group has been attached by an ester bond to a carboxylic acid at the 4-position of an antibiotic Cefditoren having a wide antimicrobial spectrum, namely (+)-(6R,7R)-7-[(Z)-2-(2-aminothiazol-4-yl)-2-methoxyiminoacetamido]-3-[(Z-)-2-(4-methylthiazol-5-yl)ethenyl]-8-oxo-5-thia-1-azabicyclo[4.2.0]oct-2-e- ne-2-carboxylic acid, known also in the chemical name of [6R-[3(Z),6.alpha.,7.beta.(Z)]]-7-[[(2-amino-4-thiazolyl)(methoxyimino)ac- etyl]amino]-3-[2-(4-methyl-5-thiazolyl)ethenyl]-8-oxo-5-thia-1-azabicyclo[-4.2.0]oct-2-ene-2-carboxylic acid, for the purpose of imparting excellent oral absorption to Cefditoren.

Crystalline Cefditoren pivoxil is known to have high purity, high heat stability and in addition satisfactory stability even when stored under high humidity conditions as disclosed in U.S. Pat. No. 6,294,669. However, crystalline Cefditoren pivoxil has low solubility in water and thus is not suitable for oral administration. Low-solubility drugs often show poor bioavailability or irregular absorption, the degree of irregularity being affected by factors, such as dose level, fed state of the patient, and form of the drug.

U.S. Pat. Nos. 6,342,493 and 6,486,149 disclose the conversion of crystalline Cefditoren pivoxil to amorphous form. In such conversion, crystalline Cefditoren pivoxil is dissolved in an acidic aqueous solution containing a water-soluble polymeric additive, the acidic aqueous solution is neutralized to coprecipitate Cefditoren pivoxil together with the water-soluble polymeric additive, and the precipitate is then collected, washed and dried. According to this method, a yellow-colored powdery composition comprising solid particles of an intimate mixture of an amorphous form of Cefditoren pivoxil having a high level of dissolvability in water and high heat stability with the water-soluble polymeric additive (0.5 to 5%) can be provided. This method, however, involves many steps and thus requires process control and is time consuming.

One of the reported methods involves the conversion of a medicinal compound that is sparingly soluble in water into an amorphous substance, thus to improve the solubility of the compound in water. Conversion of crystalline Cefditoren pivoxil to an amorphous form leads to high water solubility and improves the usefulness of Cefditoren pivoxil in the therapy of disease.

According to U.S. Patent Application No. 2008/0069879 amorphous compositions with improved bioavailability have a disadvantage: amorphous materials are thermodynamically unstable and therefore show some tendency to crystallize spontaneously. Wet granulation should be avoided, as addition of a solvent along with, and subsequent removal in way of drying the granules at elevated temperatures may covert the amorphous form to crystalline form.

U.S. Patent Application No 2004/0115272 discloses a method of conversion of crystalline Cefditoren pivoxil to amorphous form by grinding Cefditoren pivoxil in the presence of a pharmaceutically acceptable organic polymeric compound.

European Patent No. 0629404 discloses a pharmaceutical composition comprising Cefditoren pivoxil and a water soluble polymer-like hydroxypropylcellulose. This preparation was disclosed to have improved wettability, dispersibility and absorbability without increasing its bitterness. EP 0339465 discloses improved dispersibility and absorbability for a composition containing Cefditoren pivoxil and .beta.-cyclodextrin along with an ionic surfactant in a pharmaceutically acceptable carrier.

U.S. Patent Application No. 2006/0051411 discloses a pharmaceutical composition comprising amorphous Cefditoren pivoxil and a sugar ester fatty acid, which was obtained by mixing or wet-granulating particles containing amorphous Cefditoren pivoxil with the sugar ester fatty acid while amorphous Cefditoren pivoxil maintains its particle state.

Cefditoren pivoxil, apart from exhibiting low solubility, has another unfavorable property of bitterness. Cefditoren does not exhibit the bitter taste by itself upon oral administration thereof, whilst Cefditoren pivoxil exhibits a strong bitter taste on oral administration. Thus, there is a need that the bitter taste of Cefditoren pivoxil should be minimized to such an extent that the oral administration of Cefditoren pivoxil would be palatable to patients.

U.S. Pat. No. 5,958,915 dislcloses addition of a water-soluble casein salt to Cefditoren pivoxil as a method for enhancing solubility of the drug, with minimized bitter taste. This patents covers the the marketed Cefditoren Pivoxil tablets under the brand name Spectracef®. However, consumption of formulations using water-soluble casein may cause difficulties for individuals with lactose intolerance because of their inability to digest significant amounts of lactose, which is the predominant sugar.

These methods, however, involve many steps and thus require process control and a relatively lot of time. Therefore, the above method is not necessarily satisfactory from the viewpoint of production efficiency.

### OBJECTS OF THE INVENTION

The object of the invention is a pharmaceutical composition comprising Cefditoren pivoxil, water soluble high molecular weight substance and one or more pharmaceutically acceptable excipient wherein weight ratio of water soluble high molecular weight substance to Cefditoren pivoxil is greater than 1:4.

Another object is a stable pharmaceutical composition comprising Cefditoren pivoxil, water soluble high molecular weight substance, stabilizing agent and one or more a pharmaceutically acceptable excipient wherein weight ratio of water soluble high molecular weight substance to Cefditoren pivoxil is greater than 1:4.

Another object is a process of preparing a stable pharmaceutical composition of Cefditoren pivoxil comprising water soluble high molecular weight substance, stabilizing agent and one or more a pharmaceutically acceptable excipient, said process comprising the steps of mixing cefditoren pivoxil with stabilizing agents and one or more pharmaceutical excipients, granulating the said mixture using water soluble high molecular weight substance, wherein weight ratio of water soluble high molecular weight substance to cefditoren pivoxil is greater than 1:4.

Another object is a pharmaceutical composition comprising Cefditoren pivoxil, water soluble high molecular weight substance and one or more pharmaceutically acceptable excipient wherein weight ratio of water soluble high molecular weight substance to Cefditoren pivoxil is greater than 1:4 having an in-vitro dissolution profile, when measured in a Type II Paddle dissolution apparatus, in simulated gastric fluid at about 75 rpm, is, as follows (a) less than about 15% of the cefditoren pivoxil is released after about 5 mins, (b) less than about 70% of the cefditoren pivoxil is released after about 15 mins.

Another object is a pharmaceutical composition comprising Cefditoren pivoxil, water soluble high molecular weight substance and one or more pharmaceutically acceptable excipient wherein weight ratio of water soluble high molecular weight substance to Cefditoren pivoxil is greater than 1:4 having Area Under Curve (abbreviated as AUC) greater than 10,000 ng. hr/ml when measured in the in-vivo bioavailability study.

### DETAILED DESCRIPTION OF THE INVENTION:

The present invention relates to the pharmaceutical composition comprising Cefditoren pivoxil, water soluble high molecular weight substance and one or more pharmaceutically acceptable excipient wherein weight ratio of water soluble high molecular weight substance to Cefditoren pivoxil is greater than 1:4.

The content of the Cefditoren pivoxil in the pharmaceutical composition may be properly selected depending upon the dosage form. The dose may be appropriately determined depending on various conditions, for example, the age, weight, gender, disease, and severity of condition of patients. The dose effective for the treatment of infectious diseases is, for example, typically 25 to 2,000 mg/kg, preferably 50 to 1,000 mg/kg, in terms of the amount of Cefditoren pivoxil. This dose may be administered at a time daily or divided doses of several times daily.

The “water soluble high molecular weight substance” comprises but not particularly limited to water-solubilized cellulose derivatives, or pluran, carrageenan, polyvinylpyrrolidone or an arginic acid ester of polypropylene glycol.

The “water-solubilized cellulose derivatives” comprises but not limited hydroxypropylmethyl cellulose (abbreviated as HPMC), hydroxypropylmethyl cellulose phthalate (abbreviated as HPMCP), hydroxypropyl cellulose (abbreviated as HPC), methyl cellulose (abbreviated as MC), carboxymethyl cellulose calcium salt or carboxymethyl cellulose sodium salt, croscarmellose sodium. More preferably, the “water-solubilized cellulose derivatives” is HPMC, HPC, or a mixture thereof.

In another embodiment, the pharmaceutical composition comprising Cefditoren pivoxil, water soluble high molecular weight substance and one or more pharmaceutically acceptable excipient wherein weight ratio of water soluble high molecular weight substance to Cefditoren pivoxil is in the range of 1:5 to 1:20.

In another embodiment, the pharmaceutical composition comprising Cefditoren pivoxil, water soluble high molecular weight substance and one or more pharmaceutically acceptable excipient wherein weight ratio of water soluble high molecular weight substance to Cefditoren pivoxil is greater than 1:4 having an in-vitro dissolution profile, when measured in a Type II Paddle dissolution apparatus, in simulated gastric fluid at about 75 rpm, is, as follows (a) less than about 15% of the cefditoren pivoxil is released after about 5 mins, (b) less than about 70% of the cefditoren pivoxil is released after about 15 mins.

In another embodiment, the pharmaceutical composition comprising Cefditoren pivoxil, water soluble high molecular weight substance and one or more pharmaceutically acceptable excipient wherein weight ratio of water soluble high molecular weight substance to Cefditoren pivoxil is greater than 1:4 having Area Under Curve (abbreviated as AUC) greater than 10,000 ng. hr/ml when measured in the in-vivo bioavailability study.

In another embodiment, the stable pharmaceutical composition comprising Cefditoren pivoxil, water soluble high molecular weight substance, stabilizing agent and one or more a pharmaceutically acceptable excipient wherein weight ratio of water soluble high molecular weight substance to Cefditoren pivoxil is greater than 1:4.

The term “stable pharmaceutical composition” means that the Cefditoren pivoxil polymorphism remain substantially intact in the composition when stored at 40° C. temperature and 75% RH (Relative Humidity) for 2 months. The stability of Cefditoren pivoxil polymorphism can be confirmed by any known analytical method in the literature comprising but not limited to X-ray Diffractometry and Differential Scanning calorimetry (DSC).

The term “crystalline Cefditoren pivoxil” means cefditoren pivoxil in a crystalline form in which substantially all cefditoren pivoxil molecules have a regular spacial configuration. In the present invention, the crystalline Cefditoren pivoxil may be a commercially available one or alternatively, the crystalline Cefditoren pivoxil could be easily prepared by a person having ordinary skill in the art according to a method described, for example, in WO 98/12200 or U.S. Pat. No. 6,294,669.

The term “stabilizing agent” refers to an agent which maintains Cefditoren pivoxil polymorphism substantially intact in the composition when stored at 40° C. temperature and 75% RH for 2 months, wherein Cefditoren pivoxil is in an amorphous state or crystalline in nature.

The term “Stabilizing agent” comprises but not limited to mannitol, sorbitol, xylitol, glucose, dextrose, fructose, maltose, galactose, sucrose, lactose, glucose, lactulose, trehalose, cellobiose, isomalt, dextrates, maltodextrin, calcium phosphate, amino acids such as alanine, arginine, aspartic acid, asparagine, cysteine, glutamic acid, glutamine, glycine, histidine, isoleucine, leucine, lysine, methionine, phenylalanine, praline, serine, threonine, tryptophane, tyrosine and valine.

In another embodiment, the stable pharmaceutical composition comprising Cefditoren pivoxil in an amorphous state, water soluble high molecular weight substance, stabilizing agent and one or more a pharmaceutically acceptable excipient wherein weight ratio of water soluble high molecular weight substance to Cefditoren pivoxil is greater than 1:4.

The term “amorphous state” refers a solid state in which the amorphous form can be confirmed by conventional X-ray diffractometry. Specifically, a crystalline Cefditoren pivoxil exhibits a sharp diffraction peak, whereas, as with the amorphous Cefditoren pivoxil does not substantially exhibit any diffraction peak.

In another embodiment, the stable pharmaceutical composition comprising Cefditoren pivoxil, water soluble high molecular weight substance, stabilizing agent and one or more a pharmaceutically acceptable excipient wherein weight ratio of Cefditoren pivoxil to stabilizing agent in the range of from about 1:0.5 to about 1:3.

In another embodiment, the stable pharmaceutical composition comprising Cefditoren pivoxil, water soluble high molecular weight substance, stabilizing agent and one or more a pharmaceutically acceptable excipient wherein weight ratio of Cefditoren pivoxil to stabilizing agent in the range of from about 1:0.5 to about 1:3 having an in-vitro dissolution profile, when measured in a Type II Paddle dissolution apparatus, in simulated gastric fluid at about 75 rpm, is, as follows (a) less than about 15% of the cefditoren pivoxil is released after about 5 mins, (b) less than about 70% of the cefditoren pivoxil is released after about 15 mins.

In another embodiment, the stable pharmaceutical composition comprising Cefditoren pivoxil, water soluble high molecular weight substance, stabilizing agent and one or more a pharmaceutically acceptable excipient wherein weight ratio of Cefditoren pivoxil to stabilizing agent in the range of from about 1:0.5 to about 1:3 having AUC greater than 10,000 ng. hr/ml when measured in the in-vivo bioavailability study.

The pharmaceutically acceptable excipient used in the pharmaceutical compositions of invention comprise but not limited to diluents, binders, pH stabilizing agents, disintegrants, surfactants, glidants and lubricants.

The amount of excipient employed will depend upon how much active agent is to be used. One excipient(s) can perform more than one function.

Binders as used in the present invention comprises but not limited to, starches such as potato starch, wheat starch, corn starch; microcrystalline cellulose such as products known under the registered trade marks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations there of and other materials known to one of ordinary skill in the art and mixtures thereof.

Fillers or diluents, as used in the present invention comprises but not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants as used in the present invention comprises but not limited to Mg, Al, Ca or Zn stearate, polyethylene glycol, glyceryl behenate, mineral oil, sodium stearyl fumarate, stearic acid, hydrogenated vegetable oil and talc.

Glidants comprises but not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants comprises but not limited to starches; clays; celluloses; alginates; gums; cross-linked polymers, e.g., cross-linked polyvinyl pyrrolidone or crospovidone, e.g., POLYPLASDONE XL, cross-linked sodium carboxymethylcellulose or croscarmellose sodium, e.g., AC-DI-SOL from FMC; and cross-linked calcium carboxymethylcellulose; soy polysaccharides; and guar gum. Use of disintegrant according to the present invention facilitates in the release of drug in the latter stage and thereby completely releasing the drug from the dosage form.

The pharmaceutical composition may optionally contain a surface-active agent. The preferred agent is copolymers composed of a central hydrophobic chain of polyoxypropylene (poly(propylene oxide)) and polyoxyethylene (poly(ethylene oxide)) that is well known as poloxamer. However, other agents may also be employed such as dioctyl sodium sulfosuccinate (DSS), triethanolamine, sodium lauryl sulphate (SLS), polyoxyethylene sorbitan and poloxalkol derivatives, quaternary ammonium salts or other pharmaceutically acceptable surface-active agents known to one ordinary skilled in the art.

The pharmaceutical composition comprises but not limited to tablets (single layered tablets, multilayered tablets, mini tablets, bioadhesive tablets, caplets, matrix tablets, tablet within a tablet, mucoadhesive tablets, modified release tablets, pulsatile release tablets, and timed release tablets), pellets, beads, granules, sustained release compositions, capsules, microcapsules, tablets in capsules, microspheres, matrix compositions, microencapsulation.

In yet another embodiment, the pharmaceutical composition may optionally be coated wherein the coating can be film coating, sugar coating, extended release coating, enteric coating, partial enteric coating or leaky enteric coating, bioadhesive coating and other coatings known in the art. These coatings may help the pharmaceutical composition to release the drug at and for the required time. The coating comprises a hydrophilic or hydrophobic substance(s) or the combinations thereof.

The hydrophobic substance in the coating is selected from group comprising but is not limited to Ammonio methacrylate copolymers type A and B as described in USP, methacrylic acid copolymer type A, B and C as described in USP, Polyacrylate dispersion 30% as described in pH. Eur., Polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate). Poly(isodecyl methacrylate), poly(lauryl methacrylate), poly(phenyl methacrylate), poly(methyl acrylate), poly(isopropyl acrylate), poly(isobutyl actylate), poly(octadecyl acrylate), waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol distearate; glycerol monooleate, acetylated monoglycerides, tristearin, tripalmitin, cetyl esters wax, glyceryl palmitostearate, glyceryl behenate, and hydrogenated castor oil.

The hydrophilic substance in the coating is selected from the group comprising but not limited to celluloses or their salts or derivatives thereof, hydroxyethylcellulose, hydroxypropyl cellulose, hydroxypropyl methylcellulose, sodium carboxymethyl cellulose, alginic acid or their salts and derivatives thereof, carbomer (Carbopol™), polyethyleneoxide, xanthan gum, guar gum, locust bean gum, poly vinyl acetate, polyvinyl alcohol, lactose, PVA these hydrophilic polymers also act as pore forming agent.

These coating comprises one or more excipients selected from the group comprising coating agents, opacifiers, taste-masking agents, fillers, polishing agents, colouring agents, antitacking agents and the like.

The pharmaceutical composition can be coated by a wide variety of methods. Suitable methods include compression coating, coating in a fluidized bed or a pan and hot melt (extrusion) coating. Such methods are well known to those skilled in the art.

Another object is a process for preparing a stable pharmaceutical composition comprising the steps of mixing cefditoren pivoxil with stabilizing agents and one or more pharmaceutical excipients, granulating the said mixture using water soluble high molecular weight substance, wherein weight ratio of water soluble high molecular weight substance to Cefditoren pivoxil is greater than 1:4.

The pharmaceutical composition can be formed by various processes known in the art but not limited to such as by dry granulation, wet granulation, melt granulation, direct compression, double compression, extrusion spheronization, layering and the like.

The solvent(s) used in wet granulation include all the solvents well known in the art or the mixtures thereof.

The pharmaceutical composition of present invention comprising Cefditoren pivoxil can be used for treatment of bacterial infections. Bacterial infections referred to herein include for example infections caused by gram-positive bacteria such as staphylococcus and streptococcus, gram-negative bacteria such as Escherchia coli, Branhamella catarrhalis, klebsiella, Proteus, and Haemophilus influenzae, and anaerobes such as Peptostreptococcus, Propionibacterium acnes, and Bactericides. Further, pharmaceutical compositions described herein can be useful for the prophylaxis or therapy of diseases induced by gram-positive bacteria or gram-negative bacteria.

The following examples are illustrative of the present invention, and the examples should not be considered as limiting the scope of this invention in any way, as these examples and other equivalents thereof will become apparent to those versed in the art, in the light of the present disclosure, and the accompanying claims.

### BRIEF DESCRIPTION OF DRAWING

X-Ray Diffraction Charts Obtained By Measuring in X-Ray Diffractometer

FIG. 1 X-ray diffraction chart of Example 2 of this invention at intial conditions.

FIG. 2 X-ray diffraction chart of Example 2 after 2 months at 40° C. temperature and 75% RH (Relative Humidity).

### EXAMPLE 1

| | | Quantity |
|---|---|---|
| | | Per Tablet |
| Sr. No. | Ingredients | in mg |
| 1 | Cefditoren pivoxil | 400 |
| 2 | HPMC | 0.4 |
| 3 | Water | q.s. |

Procedure:
1. Granulate the cefditoren pivoxil with binder solution of HPMC.
2. Dry the granules of step 1.
3. Compress the blend in step 2 by using suitable tooling.
4. Coat the tablets with the coating solution.

### EXAMPLE 2

| | | |
|---|---|---|
| | | Quantity |
| | | Per Tablet |
| Sr. No. | Ingredients | in mg |

Intragranular Excipients

| | | |
|---|---|---|
| 1 | Cefditoren pivoxil | 400 |
| 2 | Croscarmellose Sodium | 11 |
| 3 | Mannitol | 677 |
| 4 | Colloidal silicon dioxide | 10 |
| 5 | HPMC | 55 |
| 6 | Magnesium Stearate | 35 |
| 7 | Water | q.s. |

Procedure:
1. Dry mix Cefditoren pivoxil, Croscarmellose Sodium, Colloidal silicon dioxide and Mannitol.
2. Granulate the blend of step 1 with binder solution of HPMC.
3. Dry the granules of step 2.
4. Add the Magnesium starate to step 3 and mix well.
5. Compress the blend in step 4 by using suitable tooling.
6. Coat the tablets with the coating solution.

In-Vitro Dissolution Study:

The in-vitro test used to measure release rate of the active agent from the pharmaceutical composition of the invention is as follows:

A solution of 900 ml of a simulated gastric fluid and the apparatus USP Dissolution Apparatus Type II. The tablet composition was placed in the apparatus and dissolution was periodically measured. The in-vitro dissolution studies of Example 2 is such that less than about 15% of the cefditoren pivoxil is released after about 5 mins, (b) less than about 70% of the cefditoren pivoxil is released after about 15 mins.

Cumulative % Drug Release

| Time in Minutes | Example 2 | Spectracef ® |
|---|---|---|
| 0 | 0 | 0 |
| 5 | 3.8 | 26.1 |
| 10 | 25.8 | 59.9 |
| 15 | 47.4 | 81.6 |
| 20 | 63.1 | 94.1 |
| 30 | 87 | 99.2 |

In-Vivo Bioavailability Study:

Open label, balanced, randomised, single-dose, two-treatment, two-sequence, two-period crossover oral bioequivalence study of Cefditoren Pivoxil tablets 400 mg of Lupin Limited, India comparing with that of Spectracef® (Containing Cefditoren 400 mg) tablets distributed by Cornerstone Biopharma, Inc Cary, N.C. 27518 in 12 healthy, adult, male, human subjects under fasting and fed conditions.

The study was designed to demonstrate the similar clinical efficacy compared to Spectracef®. The in-vivo bioavailability study in fasting and fed state shows the results as shown in the tables below:

TABLE 1
Comparative pharmacokinetic parameters of Example 2 vs
Spectracef ® in Fasted state

| | AUC (0-t) | AUC (0-∞) | Cₘₐₓ |
|---|---|---|---|
| | ng · hr/ml | ng · hr/ml | ng/ml |
| Example 2 (T) | 18914 | 19751 | 5453 |
| Spectracef 400 mg (R) | 15871 | 16444 | 4728 |
| T/R | 114.75 | 117.92 | 106.19 |

TABLE 2
Comparative pharmacokinetic parameters of Example 2 vs
Spectracef ® in Fed state

| | AUC (0-t) | AUC (0-∞) | Cₘₐₓ |
|---|---|---|---|
| | ng · hr/ml | ng · hr/ml | ng/ml |
| Example 2 (T) | 29640 | 30941 | 8873 |
| Spectracef 400 mg (R) | 33782 | 34777 | 9648 |
| T/R | 86.65 | 86.66 | 90.95 |

X-RD Stability Study

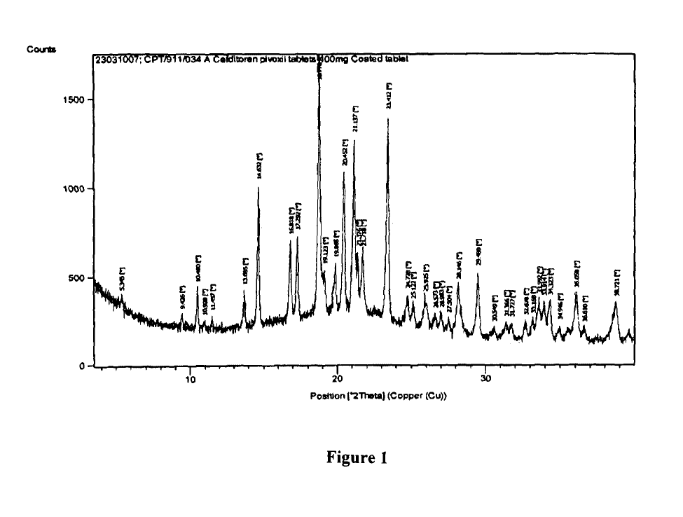

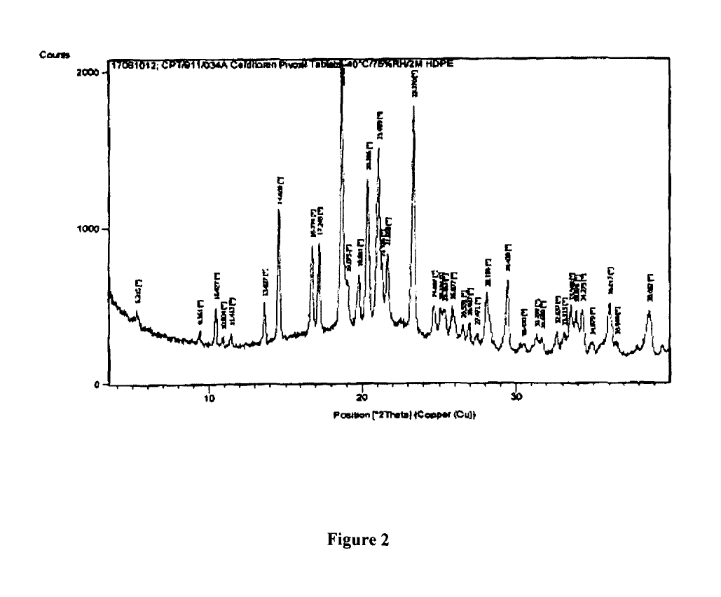

The pharmaceutical composition of Example 2 was kept for stability study at 40° C. temperature and 75% RH (Relative Humidity) conditions for 2 months. The pattern of X-ray diffraction for the pharmaceutical composition of Example 2 obtained by measuring in X-ray diffractometer is shown in FIGS. 1 and 2 and found to be stable.

1. A pharmaceutical composition comprising Cefditoren pivoxil, water soluble high molecular weight substance and one or more pharmaceutically acceptable excipient wherein weight ratio of high molecular weight substance to Cefditoren pivoxil is greater than 1:4.

2. The pharmaceutical composition of claim 1 wherein the water soluble high molecular weight substance is selected from water-solubilized cellulose derivatives, pluran, carrageenan, polyvinylpyrrolidone and an arginic acid ester of polypropylene glycol or combinations thereof.

3. The pharmaceutical composition of claim 1 wherein one or more pharmaceutically acceptable excipient is selected from binders, diluents, lubricants, surfactants or glidants.

4. A pharmaceutical composition comprising Cefditoren pivoxil, water soluble high molecular weight substance and one or more pharmaceutically acceptable excipient wherein weight ratio of high molecular weight substance to Cefditoren pivoxil is greater than 1:4 having an in-vitro dissolution profile, when measured in a Type II Paddle dissolution apparatus, in simulated gastric fluid at about 75 rpm, is, as follows (a) less than about 15% of the cefditoren pivoxil is released after about 5 mins, (b) less than about 70% of the cefditoren pivoxil is released after about 15 mins.

5. A stable pharmaceutical composition comprising Cefditoren pivoxil, water soluble high molecular weight substance, stabilizing agent and one or more a pharmaceutically acceptable excipient wherein weight ratio of water soluble high molecular weight substance to Cefditoren pivoxil is greater than 1:4.

6. The stable pharmaceutical composition of claim 5 wherein weight ratio of Cefditoren pivoxil to stabilizing agent is in the range of from about 1:0.5 to about 1:3.

7. The pharmaceutical composition of claim 5 wherein stabilizing agent is selected from mannitol, sorbitol, xylitol, glucose, dextrose, fructose, maltose, galactose, sucrose, lactose, glucose, lactulose, trehalose, cellobiose, isomalt, dextrates, maltodextrin, calcium phosphate, amino acids such as alanine, arginine, aspartic acid, asparagine, cysteine, glutamic acid, glutamine, glycine, histidine, isoleucine, leucine, lysine, methionine, phenylalanine, praline, serine, threonine, tryptophane, tyrosine and valine or mixtures thereof.

8. The stable pharmaceutical composition of claim 5 having an in-vitro dissolution profile, when measured in a Type II Paddle dissolution apparatus, in simulated gastric fluid at about 75 rpm, is, as follows (a) less than about 15% of the cefditoren pivoxil is released after about 5 mins, (b) less than about 70% of the cefditoren pivoxil is released after about 15 mins.

9. A process of preparing a stable pharmaceutical composition of Cefditoren pivoxil comprising water soluble high molecular weight substance, stabilizing agent and one or more a pharmaceutically acceptable excipient, said process comprising the steps of mixing cefditoren pivoxil with stabilizing agents and one or more pharmaceutical excipients, granulating the said mixture using water soluble high molecular weight substance, wherein weight ratio of water soluble high molecular weight substance to cefditoren pivoxil is greater than 1:4.

10. A pharmaceutical composition of claim 1 adapted for treating a disease induced by gram-positive bacteria or gram-negative bacteria.
